(19) United States
(12) Patent Application Publication
(10) Pub. No.: US 2025/0163356 A1
(43) Pub. Date: May 22, 2025

(54) SYSTEMS AND METHODS FOR REJUVENATING CELL CULTURE MEDIUM

(71) Applicant: FUTURE MEAT TECHNOLOGIES LTD., Rehovot (IL)

(72) Inventors: Yaakov NAHMIAS, Rehovot (IL); Gidon HALAF, Rehovot (IL); Zach SHIDLOVSKY, Rehovot (IL); Hila ZUKERMAN NARODIZKY, Rehovot (IL)

(73) Assignee: FUTURE MEAT TECHNOLOGIES LTD., Rehovot (IL)

(21) Appl. No.: 18/839,125

(22) PCT Filed: Feb. 16, 2023
(86) PCT No.: PCT/IB2023/051415

§ 371 Date: Aug. 16, 2024

Related U.S. Application Data
(60) Provisional application No. 63/310,654, filed on Feb. 16, 2022.

(51) Int. Cl. C12M 1/00; A23L 13/00; B01D 21/26; B01D 61/02; B01D 61/04; B01D 61/44; B01D 61/58; C12M 1/26; C12N 5/077

(52) U.S. Cl. CPC C12M 29/26; A23L 13/00; B01D 21/262; B01D 61/58; C12M 29/04; C12M 29/16; C12M 33/10; C12N 5/0658; B01D 61/027; B01D 61/04; B01D 61/445; B01D 2311/04

(57) ABSTRACT

The present disclosure provides, in part, a system for rejuvenating a cell culture medium. Also provided is a method for rejuvenating a cell culture medium. Such system and method can be used to produce culture meat.

### CROSS REFERENCE TO RELATED APPLICATIONS

This application claims the benefit of the U.S. Provisional application No. 63/310,654 filed Feb. 16, 2022, the disclosures of which is herein incorporated by reference in its entirety.

### FIELD

The present disclosure generally relates to medium rejuvenation. More specifically, the present disclosure relates to a system and a method for rejuvenating a cell culture medium as well as methods of expanding cells in the medium and thereby producing cultured meat.

### BACKGROUND

Animal cells produce waste byproducts during their expansion. Waste products such as ammonia and lactate actively inhibit the expansion of cells thus limiting biomanufacturing processes. Continuous culturing is one way of removing waste products during culturing, wherein a culture medium containing waste products is actively removed from a bioreactor in a process termed perfusion, and a fresh medium is continuously added. Several devices exist that allow effective cell retention for continuous culturing, which include acoustic and rotational filter, tangential flow filtration devices, and inclined settlers. However, perfusion is an expensive process as waste medium is continuously discarded before nutrients are depleted.

Over the years, devices were developed permitting removal of waste products from a culture medium and its recirculation in a process termed rejuvenation. To date, these medium rejuvenation devices rely on absorption or filtration. The process of waste product absorption relies on resins that can bind to ammonia or lactate, while filtration allows the waste products to pass through membranes and hence, the medium retains critical nutrients. Both these processes require the a priori removal of large proteins from the culture medium using low-capacity ultrafiltration, which limit the size of bioreactors supported by medium rejuvenation.

### SUMMARY

The present disclosure provides, in part, systems and methods for separating essential materials from waste materials in a liquid medium, thus rejuvenating the medium for continuous use. While the systems or methods may be used for treating a vast array of liquid formulations or compositions, the present disclosure focuses on using these systems as efficient and simple ways to separate waste components from essential components of cell culture media and recycle the media for continuous use.

Accordingly, one embodiment of the present disclosure provides a system for rejuvenating a cell culture medium. Such system comprises (a) means for obtaining a cell culture medium comprising waste molecules and essentially devoid of cells from a bioreactor using a cell retention device; (b) means for separating charged molecules from the cell culture medium obtained from (a) using an electric field, thereby removing the waste molecules from the cell culture medium and obtaining a rejuvenated medium essentially devoid of waste molecules; (c) means for retaining uncharged molecules and macromolecules in the rejuvenated medium during separation and removal of the charged molecules; and (d) means for circulating the rejuvenated medium back into the bioreactor.

In one embodiment, the current disclosure encompasses a system for rejuvenating a cell culture medium, the system comprising: (a) means for obtaining a cell culture medium essentially devoid of cells from at least one bioreactor using a cell retention device, wherein the cell culture medium comprises one or more waste molecule; (b) a nanofiltration means in fluid communication with the means in (a), wherein the nanofiltration means is operable to provide a filtered permeate reduced in the one or more waste molecules; (c) means for separating charged molecules from the filtered permeate obtained from (b) using an electric field, thereby further reducing the one or more waste molecules from the cell culture medium and obtaining a rejuvenated medium essentially devoid of the one or more waste molecules; (e) means for retaining uncharged molecules and macromolecules in the rejuvenated medium during separation and removal of the charged molecules; and (f) means for circulating the rejuvenated medium back into the at least one bioreactor or into at least one other bioreactor.

In some aspects, the current disclosure encompasses a system for efficient growth of eucaryotic cells for medical or agriculture applications, the system comprising: (a) at least one means for growing eucaryotic cells, such as a bioreactor, connected to a cell retention centrifuge, producing a waste medium essentially devoid of cells, wherein the waste medium is a cell culture medium comprising one or more waste molecules; (b) means for collecting the waste medium and transferring the waste medium to a rejuvenation tank; (c) a means for controlling the pH of the waste medium from (b) prior to nanofiltration; (d) a means for nanofiltration of the waste medium from (b), thereby producing a permeate and a concentrated waste stream; (e) a means of removal of one or more waste molecules, such as electrodialysis (ED), from the permeate, thereby producing a polished rejuvenation medium; (f) a means for recycling the concentrated waste or the polished rejuvenation medium or both back to the at least one means for growing eucaryotic cells, thereby increasing the efficient use of culture medium for eucaryotic cell growth.

In some embodiments, the cell culture medium obtained from (a) of any of the above systems may further comprise one or more proteins.

In some embodiments, the waste molecules interfere with desired growth and/or desired differentiation of the cells, which include, but are not limited to, ammonia, ammonium, lactate, sodium, isovaleric acid, isobutyric acid, 2-methylbutyric acid, rosmarinic acid, 3-phenyllactic acid, DL-p-Hydroxyphenyllactic acid, fumarate, alanine, glutamic acid, aspartic acid, reactive oxygen and nitrogen species, or a combination thereof. In some particular embodiments, the waste molecules may include ammonia, ammonium, and/or lactate.

In some embodiments, the rejuvenated medium contains one or more proteins selected from the group consisting of albumin, catalase, insulin, fibroblast growth factor, transforming growth factor beta, and super oxide dismutase.

In some embodiments, the cell retention device may comprise at least one hollow fiber with a pore cutoff between about 5 kD and about 5 microns. By way of non-limiting example, the at least one hollow fiber has a pore cutoff of about 3 microns. In some embodiments, the cell retention device comprises continuous or non-continuous centrifuge. By way of non-limiting example, the centrifuge may operate at 600 to 20,000×g, in some particular embodiments, the centrifuge may operate at 8400×g, thereby removing cells from the cell culture medium and creating a waste medium devoid of cells for rejuvenation.

In some embodiments, the means for separating charged waste molecules from the cell culture medium may comprise an electrodialysis (ED) unit. In some embodiments, the ED unit may be a standard ED or a bi-polar ED (BPED). BPED comprises a bi-polar membrane which allows for separate recovery of lactic acid and ammonium.

In some embodiments, the ED unit may employ a voltage in the range of about 5-20 Volt for a 10-membranes stack (10 anions & 11 cations for standard ED, or 11 cations & 10 anions & 10 BiPolar membranes for BPED) on the cell culture medium. By way of non-limiting example, the ED unit may employ about 0.1-4 Amperes on the cell culture medium depending on the osmolarity.

In some embodiments, the cell culture medium has a pH value that is higher than the pKa of lactate (3.8) while going through ED. In some embodiments, the cell culture medium has a pH value in the range of about 6-8 while going through ED. By way of non-limiting example, the cell culture medium has a pH value of about 7.

In some embodiments, the rejuvenated medium comprising salts and metals may be further processed at a pH in the range of about 6-8. In some embodiments, the rejuvenated medium comprises iron and zinc adjusted to at least 0.05 mg/L before returning to the bioreactor.

In some embodiments, any of the systems described above and herein may further comprise a conductivity sensor to measure the osmolarity of the rejuvenated medium.

In some embodiments, the osmolarity of the rejuvenated medium is adjusted to be about 280-320 milliosmoles per kilogram (mOsm/kg) before returning to the bioreactor. By way of non-limiting example, the rejuvenated medium has an osmolarity of about 290 mOsm/kg.

Any of the systems described above and herein may be used for production of cultured meat, glycosylated proteins, viruses, genetic materials, or vaccines.

Another embodiment of the present disclosure provides a method for rejuvenating a cell culture medium. Such method may comprise (a) obtaining a cell culture medium comprising waste molecules and essentially devoid of cells from a bioreactor using a cell retention device; (b) separating charged molecules from the cell culture medium obtained from (a) using an electric field, thereby removing the waste molecules from the cell culture medium and obtaining a rejuvenated medium essentially devoid of waste molecules; (c) retaining uncharged molecules and macromolecules in the rejuvenated medium during separation and removal of the waste molecules; and (d) circulating the rejuvenated medium back into the bioreactor, thereby rejuvenating the cell culture medium.

In some embodiments, the current disclosure also encompasses a method for rejuvenating a cell culture medium, the method comprising: (a) obtaining a cell culture medium essentially devoid of cells from at least one bioreactor using a cell retention device, wherein the cell culture medium comprises one or more waste molecules; (b) passing the cell culture medium from (a) through a nanofiltration mean, wherein the nanofiltration means is operable to provide a filtered permeate reduced in the one or more waste molecules; (c) separating charged molecules from the filtered permeate obtained from (b) using an electric field, thereby further reducing the one or more waste molecules from the cell culture medium and obtaining a rejuvenated medium essentially devoid of the one or more waste molecules; (d) retaining uncharged molecules and macromolecules in the rejuvenated medium during separation and removal of the one or more waste molecules; and (e) circulating the rejuvenated medium back into the at least one bioreactor or at least one other reactor, thereby rejuvenating the cell culture medium.

In one embodiment, the current disclosure encompasses a method for efficient growth of eucaryotic cells for medical or agriculture applications, the method comprising: (a) obtaining a cell culture medium essentially devoid of cells from at least one bioreactor using a cell retention centrifuge, thereby producing a waste medium essentially devoid of cells, wherein the waste medium is a cell culture medium comprising one or more waste molecules; (b) collecting the waste medium and transferring the waste medium to a rejuvenation tank; (c) controlling the pH of the waste medium from (b) prior to nanofiltration; (d) passing the cell culture medium from c) through a nanofiltration mean, thereby producing a permeate and a concentrated waste stream; (e) removing one or more waste molecules from the permeate using an electric field, thereby producing a polished rejuvenated medium; (f) recycling the concentrated waste or the polished rejuvenation medium or both back to the at least one means for growing eucaryotic cells, thereby increasing the efficient use of culture medium for eucaryotic cell growth.

In some embodiments, the cell culture medium obtained from (a) may further comprise one or more proteins.

In some embodiments, the waste molecules may interfere with desired growth and/or desired differentiation of the cells, which include, but are not limited to, ammonia, ammonium, lactate, sodium, isovaleric acid, isobutyric acid, 2-methylbutyric acid, rosmarinic acid, 3-phenyllactic acid, DL-p-Hydroxyphenyllactic acid, fumarate, alanine, glutamic acid, aspartic acid, reactive oxygen and nitrogen species, or a combination thereof. By way of non-limiting example, the waste molecules may comprise ammonia, ammonium, and/or lactate.

In some embodiments, the rejuvenated medium may contain one or more proteins selected from the group consisting of albumin, catalase, insulin, fibroblast growth factor, transforming growth factor beta, and super oxide dismutase.

In some embodiments, the cell retention device may comprise at least one hollow fiber with a pore cutoff between about 5 kD and about 5 microns. By way of non-limiting example, the at least one hollow fiber has a pore cutoff of about 3 microns. In some embodiments, the cell retention device may comprise continuous or non-continuous centrifuge. By way of non-limiting example, the centrifuge may operate at 600 to 20,000×g, in some particular embodiments, the centrifuge may operate at 8400×g, thereby removing cells from the cell culture medium and creating a waste medium devoid of cells for rejuvenation. In some embodiments, the nanofiltration means has a molecular weight cutoff in the range of about 150 Da to about 300 Da.

In some embodiments, the means for separating charged waste molecules from the cell culture medium may comprise an electrodialysis (ED) unit. In some embodiments, the ED unit may be a standard ED or a bi-polar ED (BPED). BPED comprises a bi-polar membrane which allows for separate recovery of lactate and ammonium.

In some embodiments, the ED unit may employ a voltage in the range of about 5-30 Volts for a 10-stack membrane on the cell culture medium. By way of non-limiting example, the ED unit may employ about 0.1-4 amperes on the cell culture medium.

In some embodiments, the cell culture medium may have a pH value in the range of about 6-8 while going through the ED process. By way of non-limiting example, the cell culture medium may have a pH value of about 7.

In some embodiments, the rejuvenated medium comprising salts and metals may be further processed at a pH in the range of about 6-8. In some embodiments, the rejuvenated medium comprises iron and zinc adjusted to at least 0.05 mg/L before returning to the bioreactor.

In some embodiments, any of the methods described above and herein may further comprise measuring the osmolarity of the rejuvenated medium by a conductivity sensor.

In some embodiments, the osmolarity of the rejuvenated medium may be adjusted to be about 280-320 milliosmoles per kilogram (mOsm/kg) before returning to the bioreactor. By way of non-limiting example, the rejuvenated medium may have an osmolarity of about 290 mOsm/kg.

For any of the methods described above and herein, the rejuvenated cell culture medium may be used to produce cultured meat, glycosylated proteins, viruses, genetic materials, or vaccines.

Some embodiments of the present disclosure provide a method for expanding cells in a bioreactor. This method may comprise culturing cells or tissues in a cell culture medium comprising nutrients and waste molecules; and rejuvenating the cell culture medium according to any of the methods disclosed above and herein to reduce the amount of waste molecules or remove the waste molecules from the medium. In some embodiments, the expanded cells are used to produce cultured meat, glycosylated proteins, viruses, genetic materials, or vaccines.

In some embodiments, the current disclosure encompasses a rejuvenated cell culture medium, wherein the rejuvenated cell culture medium is obtained from a system as disclosed herein. In some embodiments, the current disclosure also encompasses a rejuvenated cell culture medium, wherein the rejuvenated cell culture medium is obtained from any of the methods disclosed herein.

In some embodiments, the current disclosure also encompasses a cultured cell, wherein the cultured cell is obtained by culturing a cell in a rejuvenated cell culture medium obtained from any of the methods disclosed herein.

In some embodiments, the cultured cell is obtained by culturing a cell in any of the systems as disclosed herein.

In some embodiments, the current disclosure encompasses a cultured meat comprising cultured cells, wherein the cultured cells are obtained by culturing cells in a system disclosed herein.

In some embodiments, the current disclosure encompasses a cultured meat comprising cultured cells, wherein the cultured cells are grown in rejuvenated cell culture medium obtained from a method disclosed herein.

In some embodiments, the current disclosure encompasses a cultured cell, wherein the cultured cell has been obtained by a method, wherein the method comprises: (a) obtaining a cell culture medium essentially devoid of cells from at least one bioreactor using a cell retention device, wherein the cell culture medium comprises one or more waste molecules; (b) separating charged molecules from the cell culture medium obtained from (a) using an electric field, thereby removing the one or more waste molecules from the cell culture medium and obtaining a rejuvenated medium essentially devoid of the one or more waste molecules; (c) retaining uncharged molecules and macromolecules in the rejuvenated medium during separation and removal of the one or more waste molecules; (d) circulating the rejuvenated medium back into the at least one bioreactor or at least one other bioreactor; and (e) culturing the cell in the rejuvenated medium, thereby obtaining the cultured cell.

In some embodiments, the current disclosure also encompasses a cultured cell, wherein the cultured cell has been obtained by a method, wherein the method comprises: (a) obtaining a cell culture medium essentially devoid of cells from at least one bioreactor using a cell retention device, wherein the cell culture medium comprises one or more waste molecules; (b) passing the cell culture medium from (a) through a nanofiltration mean, wherein the nanofiltration means is operable to provide a filtered permeate reduced in the one or more waste molecules; (c) separating charged molecules from the filtered permeate obtained from (b) using an electric field, thereby further reducing the one or more waste molecules from the cell culture medium and obtaining a rejuvenated medium essentially devoid of the one or more waste molecules; (d) retaining uncharged molecules and macromolecules in the rejuvenated medium during separation and removal of the one or more waste molecules; (e) circulating the rejuvenated medium back into the at least one bioreactor or at least one other reactor; and (f) culturing the cell in the rejuvenated medium, thereby obtaining the cultured cell.

In some embodiments, the current disclosure further encompasses a cultured cell, wherein the cultured cell has been produced in a system, wherein the system comprises: (a) at least one bioreactor comprising or configured to comprise one or more cells; (b) means for obtaining a cell culture medium essentially devoid of cells from the bioreactor using a cell retention device, wherein the cell culture medium comprises one or more waste molecules; (c) means for separating charged molecules from the cell culture medium obtained from (a) using an electric field, thereby removing the one or more waste molecules from the cell culture medium and obtaining a rejuvenated medium essentially devoid of one or more waste molecules; (d) means for retaining uncharged molecules and macromolecules in the rejuvenated medium during separation and removal of the charged molecules; and (e) means for circulating the rejuvenated medium back into the at least one bioreactor comprising or configured to comprise one or more cells or at least one other bioreactor comprising or configured to comprise one or more cells, thereby operable to produce the cultured cell.

In some embodiments, the current disclosure also encompasses a cultured cell, wherein the cultured cell has been produced in a system, wherein the system comprises: (a) at least one bioreactor; (b) means for obtaining a cell culture medium essentially devoid of cells from the bioreactor using a cell retention device, wherein the cell culture medium comprises one or more waste molecules; (c) a nanofiltration means in fluid communication with the means in (a), wherein the nanofiltration means is operable to provide a filtered permeate reduced in the one or more waste molecules; (d) means for separating charged molecules from the filtered permeate obtained from (b) using an electric field, thereby further reducing the one of more waste molecules from the cell culture medium and obtaining a rejuvenated medium essentially devoid of one or more waste molecules; (e) means for retaining uncharged molecules and macromolecules in the rejuvenated medium during separation and removal of the charged molecules; and (f) means for circulating the rejuvenated medium back into the at least one bioreactor comprising or configured to comprise one or more cells or at least one other bioreactor comprising or configured to comprise one or more cells, thereby operable to produce the cultured cell.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic representation of a standard electrodialysis (ED).

FIG. 1B is a schematic representation of a bi-polar electrodialysis (BPED). CEM: cation exchange membrane; AEM: anion exchange membrane; BM: bi-polar membrane; A⁻: an anion, M⁺: a cation.

FIG. 2A is a schematic diagram of the rejuvenation system based on ED with common waste stream. (1) is a bioreactor, (2) is a cell retention device, (3) is an ED unit, (4) is a waste stream and (5) is rejuvenated medium.

FIG. 2B is a schematic diagram of the rejuvenation system based on BPED. (1) is a bioreactor, (2) is a cell retention device, (3) is a BPED unit, (4a) is a base waste stream, (4b) is an acid waste stream and (5) is rejuvenated medium.

FIG. 2C illustrates a further treatment for lactate and ammonium recovery. (5) is an ED unit (6) is a BPED unit, (7) is a scrubber and (8) is a extraction unit.

FIG. 2D is a schematic diagram of the rejuvenated system based on nanofiltration process and nanofiltration permeate polishing by standard ED. (201) is a bioreactor, (202) is a cell retention device, (203) is pre-nanofiltration holding tank, (204) is a nanofiltration unit, (205) is a ED unit, (206) is a concentrate stream, (207) is a diluate stream and (208) is a rejuvenated stream.

FIG. 3A illustrate reduction measurement of lactate using ED with applied voltage of 8 volts (circles), 12 volts (squares) and 14 volts (triangles). The experiments were carried out using PC-SK as a cation exchange membrane (CEM) and PC 100 D (filled shapes) or PC 200 D (blank shapes) as an anion exchange membrane (AEM).

FIG. 3B illustrate reduction measurement of glutamate using ED with applied voltage of 8 volts (circles), 12 volts (squares) and 14 volts (triangles). The experiments were carried out using PC-SK as a cation exchange membrane (CEM) and PC 100 D (filled shapes) or PC 200 D (blank shapes) as an anion exchange membrane (AEM).

FIG. 3C illustrate reduction measurement of glutamine using ED with applied voltage of 8 volts (circles), 12 volts (squares) and 14 volts (triangles). The experiments were carried out using PC-SK as a cation exchange membrane (CEM) and PC 100 D (filled shapes) or PC 200 D (blank shapes) as an anion exchange membrane (AEM).

FIG. 3D illustrate reduction measurement of glucose using ED with applied voltage of 8 volts (circles), 12 volts (squares) and 14 volts (triangles). The experiments were carried out using PC-SK as a cation exchange membrane (CEM) and PC 100 D (filled shapes) or PC 200 D (blank shapes) as an anion exchange membrane (AEM).

FIG. 3E illustrate reduction measurement of ammonium using ED with applied voltage of 8 volts (circles), 12 volts (squares) and 14 volts (triangles). The experiments were carried out using PC-SK as a cation exchange membrane (CEM) and PC 100 D (filled shapes) or PC 200 D (blank shapes) as an anion exchange membrane (AEM).

FIG. 3F illustrate reduction measurements of osmolarity using ED with applied voltage of 8 volts (circles), 12 volts (squares) and 14 volts (triangles). The experiments were carried out using PC-SK as a cation exchange membrane (CEM) and PC 100 D (filled shapes) or PC 200 D (blank shapes) as an anion exchange membrane (AEM).

FIG. 4A illustrate the reduction rate of lactate concentration (normalized to the initial concentration) as a function of the applied voltage.

FIG. 4B illustrates the reduction rate coefficient of the ammonium concentration (normalized to the initial concentration) as a function of the applied voltage. All CEM used were PC-SK, while the AEM used were either PC 100 D (filled circles) or PC 200 D (blank circles).

FIG. 5A illustrate reduction measurement of lactate using BPED with applied voltage of 16 volts. The experiments were carried out using PC-SK as a CEM and PC acid 60 as an AEM.

FIG. 5B illustrate reduction measurement of ammonium using BPED with applied voltage of 16 volts. The experiments were carried out using PC-SK as a CEM and PC acid 60 as an AEM.

FIG. 6 shows the quantification of bovine serum albumin (BSA) in waste medium (before ED) and in rejuvenated medium (after ED) during the BPED process.

FIG. 7 shows cell proliferation in standard culture medium (control—“Ctrl”), waste medium containing 40 mM lactate and 3 mM ammonia (“lac 40 mM, NH3 3 mM”), rejuvenated medium after BPED (“lac 40 mM, NH3 3 mM after ED”), and rejuvenated medium corrected for osmolarity and salts (1:1).

FIG. 8A illustrates the reduction of the lactate originated in bioreactor waste media (filled shapes) and NF's permeate (blank shapes), using PC 200 D. All CEM used were PC-SK. The experiments were carried at voltage of 18-20V and pH 7.0.

FIG. 8B illustrates the reduction of the lactate originated in bioreactor waste media (filled shapes) and NF's permeate (blank shapes) using PC 100 D as an AEM. All CEM used were PC-SK. The experiments were carried at voltage of 18-20V and pH 7.0.

FIG. 9 illustrates reduction of lactate and ammonium at rejuvenated treatment based on nanofiltration (filled) and combined treatment of nanofiltration and ED (blank).

FIG. 10 shows amino acid concentration for rejuvenation treatment based on nanofiltration (black bars) and combined treatment of nanofiltration and ED (blank bars).

FIG. 11 shows cell proliferation in bioreactor waste medium (black bars), rejuvenated medium treated by only nanofiltration (blank bars) and rejuvenated medium treated by combined treatment of nanofiltration and standard ED (gray bars).

### DETAILED DESCRIPTION

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terminology used herein is for the purpose of describing particular embodiments only and is not intended to be limiting of the invention.

As used herein, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, “devoid of,” “free” (as in “protein free”), “essentially devoid of,” or “essentially free”, means non-detectable or a small or insignificant amount of a contaminant. The term “non-detectable” is understood as based on standard methodologies of detection known in the art at the time of this application. In some embodiments, “a small amount” refers to less than 1% by weight.

As used herein, the terms “waste material(s)”, “waste molecule(s)”, and “waste product(s)” are interchangeable. These are any materials/molecules/products that interfere with desired growth and/or desired differentiation of the cells that are cultured in a cell culture medium, e.g., inhibit cell growth and/or differentiation or induce cell death. These materials/molecules/products are usually selected amongst minerals (mainly sodium salts) and small molecules (low molecular weight molecules). By way of non-limiting examples, the waste materials/molecules include, but are not limited to, ammonia, ammonium, lactate, sodium, isovaleric acid, isobutyric acid, 2-methylbutyric acid, rosmarinic acid, 3-phenyllactic acid, DL-p-Hydroxyphenyllactic acid, fumarate, alanine, glutamic acid, aspartic acid, reactive oxygen and nitrogen species, or a combination thereof.

As used herein, the term “medium” or “cell culture medium” encompasses any such medium as known in the art, including cell suspensions, blood and compositions comprising ingredients of biological origin. Such media and cultures may contain cells (mammalian cells, chicken cells, crustacean cells, fish cells and other cells), blood components, nutrients, supplements and feeds, amino acids, peptides, proteins and growth factors (such as albumin, catalase, transferrin, fibroblast growth factor (FGF), and others), vitamins, polyamines, sugars, carbohydrates, lipids, nucleic acids, hormones, fatty acids, trace materials, certain salts (such as potassium salts, calcium salts, magnesium salts), as well as waste materials such as ammonia, lactate, toxins and sodium salts. The medium is typically an aqueous based solution that promotes the desired cellular activity, such as viability, growth, proliferation, differentiation of the cells cultured in the medium. The pH of a culture medium should be suitable to the organisms that will be grown. Most bacteria grow in pH 6.5-7.0 while most animal cells thrive in pH 7.2-7.4.

As used herein the term “method” or “methods” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The present disclosure provides, in part, the use of electrodialysis to actively remove waste products from a culture medium without the need for a preceding ultrafiltration step that depletes the waste medium from proteins. Electrodialysis is a robust process that uses electric field and semi-porous membranes to separate charged particles from liquid solutions. Electrodialysis is primarily used for water desalinization and removal of salts from foodstuff such as whey. Bipolar membrane electrodialysis can further separate the waste products into two streams: one where cations such as ammonia are drawn into, and the other where anions such as lactate and chloride are drawn into.

Prior methods of medium rejuvenation utilize absorption or nanofiltration. “Nanofiltration” is a membrane filtration-based method that uses nanometer sized pores through which particles smaller than 10 nanometers pass through the membrane. Both absorption and nanofiltration methods require waste media to be devoid of large proteins, such as albumin (56 kD), which adsorb to resins and denature in the low pH required for efficient nanofiltration causing the system to clog. Acidic pH is important for nanofiltration as it allows lactate to avoid repulsive charge with the membrane and permeates through it. However, such acidic pH causes proteins to denature. As such, proteins need to be separated before pH titration to be below 3. The methods require an ultrafiltration step (<50 kD) to separate proteins, resulting in slow filtration flux<5 LMS (liter/m²/hour), which in turn functionally limits the maximal size of a bioreactor that can be used in such processes.

The current method via electrodialysis rejuvenates waste medium containing proteins, thus cell retention is carried out with pores several micrometers in size (˜3 μm) or even in a continuous or non-continuous centrifuge resulting in a high filtration flux of about ˜50 LMS (liter/m²/hour). There is no need for ultrafiltration of proteins with ED, as the pH is kept between 6-8. This method allows the use of a bioreactor 10-time larger than what prior methods permitted, which in turn greatly decreases the capital investment needed to produce the same amount of product. Due to increased capacity, waste from multiple bioreactors can also flow into a single rejuvenation system.

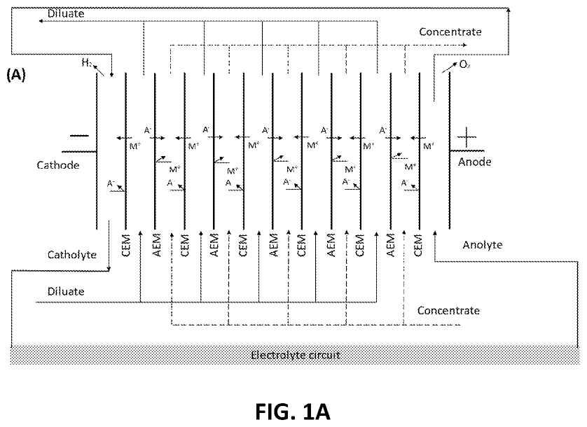

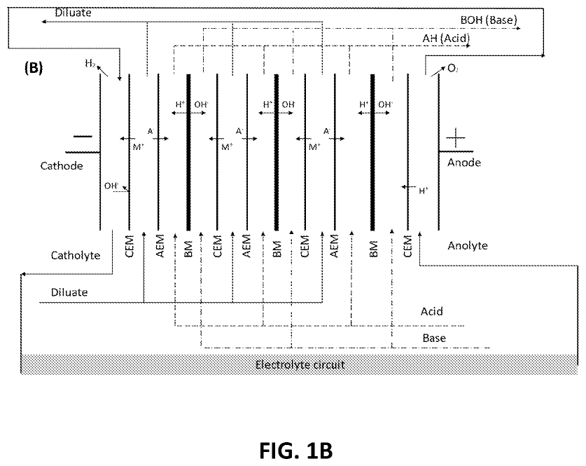

A schematic representation of a standard electrodialysis (ED) is illustrated in FIG. 1A. In the ED, an electric field is provided by a power supplier. The electric field drives the cations to the cathode side and the anions to the anode side. In the ED, there are two main streams recirculated over the cell chamber: diluate and concentrate. The diluate stream flows tangentially between a cation exchange membrane (CEM) at the cathode side and an anion exchange membrane (AEM) at the anode side. The cations (e.g., ammonium, sodium) permeate through the CEM, and anions (e.g., lactate, chloride) permeate through the AEM. Hence, the diluate stream at the outlet of the cell chamber has low concentration of charged ions (cations and/or anions). The concentrate stream also flows between CEM and AEM, but in this case the CEM located at the anode side, and AEM located in the cathode side. Hence, the concentrate stream is rich in ions that are permeated from the diluate stream. In order to supply driving force for the ED, two electrode rinse solutions are recirculated next to the cathode (catholyth) and the anode (anolyth).

A schematic representation of a bi-polar electrodialysis (BPED) is illustrated in FIG. 1B. The BPED cell chamber has additional membrane type, the bi-polar membrane (BM). Protons and hydroxyls migrate out the BM under the applied electric filed in opposite direction. The protons migrate to the cathode side, and anions that permeate form the AEM neighbor are concentrated to an acid stream (e.g., hydrochloric acid, lactic acid). The hydroxyls migrate to the anode side, and the cations that permeate from the CEM neighbor are concentrated to a base stream (e.g., sodium hydroxyl, ammonia).

That is, the standard ED has two streams: diluate and concentrate, while the BPED has three main streams: diluate, acid, and base. In the standard ED, the ions from the diluate are concentrated in a single stream (i.e., the concentrate), whereas in the BPED, the cations and the anions are separated into two different streams: the base and the acid streams, respectively. The BPED allows to recover lactic acid and ammonium separately.

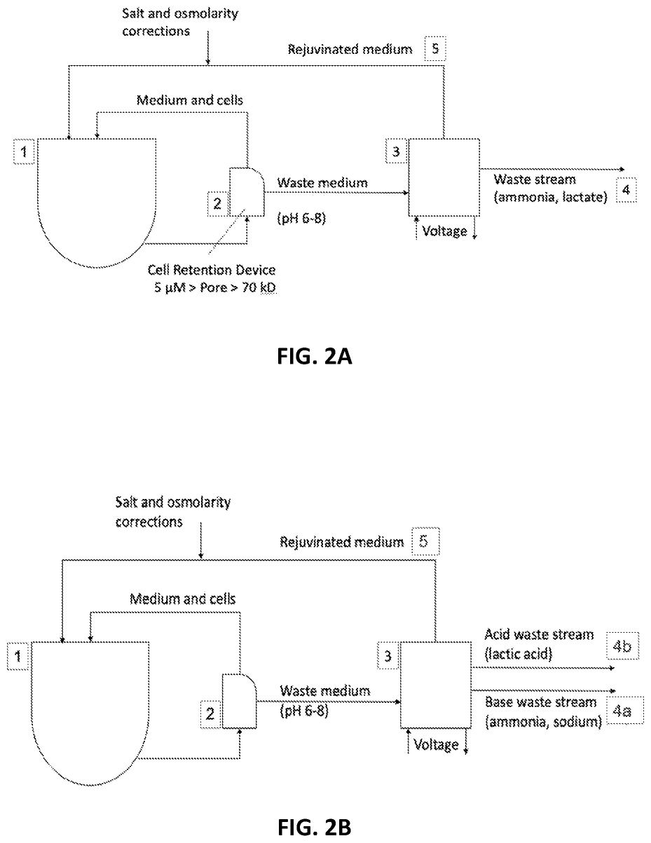

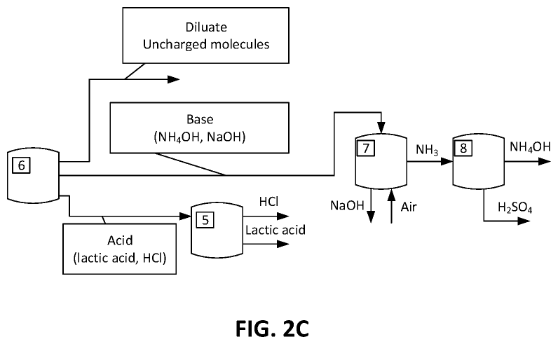

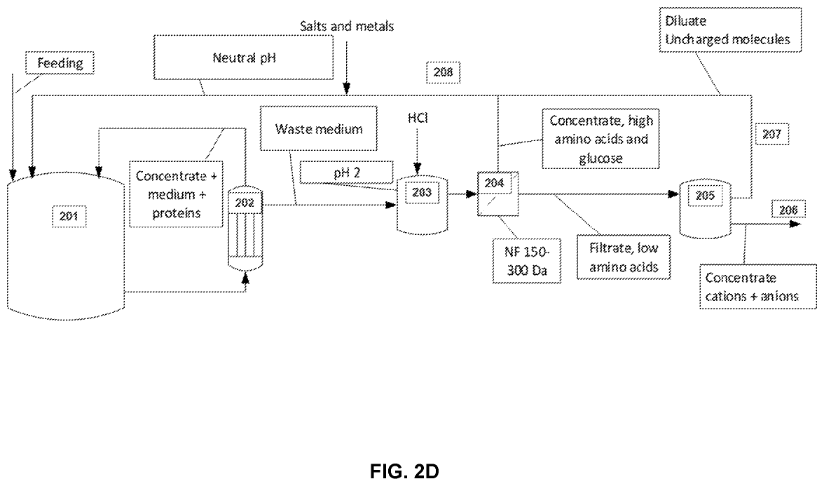

In some embodiments, the electrodialysis can be combined with nanofiltration to improve the retention of nutrients while achieving higher levels of lactate and ammonium removal. An additional rejuvenation system based on nanofiltration process and nanofiltration permeate polishing by a standard ED is illustrated in FIG. 2D. Cells are grown in a bioreactor 201 and circulated through a cell retention device 202. A waste medium containing waste products but devoid of cells is removed from the cell retention device 202 and introduced to a nanofiltration device 204, wherein the nanofiltration permeate, rich in lactate and ammonium and poor in amino acids, is introduced to an ED process 205, wherein an electric field removes ammonia, lactate and other charged waste products to an ED concentrate stream 206. Uncharged molecules remain in the ED diluate stream 207. The ED diluate 207 and the NF concentrate are merged (see, rejuvenated medium 208). The rejuvenated medium stream 208 is PH neutralized and corrected for osmolarity circulated to the bioreactor 201.

Polishing is an engineering term used to define a second action to increase the efficiency of a process. In some embodiments, rejuvenated medium with low concentrations of cell growth inhibitors like lactate is produced by nanofiltration, and is then processed through a second action, that of electrodialysis (ED) that reduces the concentration of inhibitors further, thereby “polishing” the medium.

Careful choice of semi-permeable membranes, pH, and voltage allows active removal of both ammonia and lactate at the same electrodialysis step, even in the presence of serum proteins. However, it was discovered that the process depletes positively charged metals such as iron, zinc, copper, lithium and manganese that are required for cells to grow. The process also depletes sodium and chloride ions, decreasing the osmolarity of the medium below 100 mOsm/Kg, which in turn creates a hypotonic solution that would rapidly rapture cells. An effective medium rejuvenation system would therefore consist of operating electrodialysis within narrow pH and voltage parameters while actively correcting osmolarity and metal ion concentrations in the resultant rejuvenated medium.

Provided herein are improved systems or methods of effectively remove waste molecules from cell culture media and rejuvenating the media for large scale biological manufacturing of cells, proteins, or vaccines. The systems and methods disclosed above and herein separate essential materials from waste materials in liquid media, and rejuvenating the media for continuous use, thereby provide cost effective cell culture media. While the systems or methods may be used for treating a vast array of liquid formulations or compositions, the present disclosure focuses on using these systems and methods as efficient and simple ways to separate waste molecules from essentials of cell culture media and rejuvenate the media for continuous use.

Accordingly, one embodiment of the present disclosure provides a system for rejuvenating a cell culture medium. Such system comprises (a) means for obtaining a cell culture medium comprising waste molecules and essentially devoid of cells from at least one bioreactor using a cell retention device; (b) means for separating charged molecules from the cell culture medium obtained from (a) using an electric field, thereby removing the waste molecules from the cell culture medium and obtaining a rejuvenated medium essentially devoid of waste molecules; (c) means for retaining uncharged molecules and macromolecules in the rejuvenated medium during separation and removal of the charged molecules; and (d) means for circulating the rejuvenated medium into the at least one bioreactor or at least one other bioreactor or a combinations thereof.

In one embodiment, the current disclosure also encompasses an integrated system, wherein nanofiltration and ED are combined. Therefore in one embodiment, the current disclosure encompasses a system for rejuvenating a cell culture medium, the system comprising: (a) means for obtaining a cell culture medium essentially devoid of cells from at least one bioreactor using a cell retention device, wherein the cell culture medium comprises one or more waste molecule; (b) a nanofiltration means in fluid communication with the means in (a), wherein the nanofiltration means is operable to provide a filtered permeate reduced in the one or more waste molecules; (c) means for separating charged molecules from the filtered permeate obtained from (b) using an electric field, thereby further reducing the one of more waste molecules from the cell culture medium and obtaining a rejuvenated medium essentially devoid of waste molecules; (d) means for retaining uncharged molecules and macromolecules in the rejuvenated medium during separation and removal of the charged molecules; and (e) means for re-circulating the rejuvenated medium back into the at least one bioreactor or at least one other bioreactor or combinations thereof.

In some embodiments of the systems provided herein, culture medium from multiple bioreactors can be passed through a single rejuvenation system. In some embodiments, the culture medium from at least 1, or at least 2, or at least 3, or at least 4, or at least 5, or at least 10 or more bioreactors can flow through a single rejuvenation system. Similarly, rejuvenated medium may be circulated to a single or multiple bioreactors. For example, in some embodiments, the rejuvenated media can be circulated back to at least 1, or at least 2, or at least 3, or at least 4, or at least 5, or at least 10, or more bioreactors. In some embodiments, the system may be a closed or partially closed system wherein the waste medium from one or more bioreactors is processed through (b) and (c), or (b), (c) and (d) of the integrated system and the resulting rejuvenated medium is re-circulated to the one or more bioreactors. In some embodiments the system may be an open or partially open system, wherein the rejuvenated system flows into another set of (other) one or more bioreactors. In one embodiment, the rejuvenated medium can be collected, stored or packaged for later use. In one embodiment, the rejuvenated medium is circulated to a bioreactor comprising cells or tissues.

In some embodiments, the cell culture medium obtained from (a) may further comprise one or more proteins. In some embodiments, the waste materials or molecules may be any materials or molecules that interfere with desired growth and/or desired differentiation of cells cultured in the cell culture medium. For instance, the waste materials or molecules may inhibit cell growth and/or differentiation or induce cell death. In some embodiments, the waste molecules include, but are not limited to, ammonia, ammonium, lactate, sodium, isovaleric acid, isobutyric acid, 2-methylbutyric acid, rosmarinic acid, 3-phenyllactic acid, DL-p-Hydroxyphenyllactic acid, fumarate, alanine, glutamic acid, aspartic acid, reactive oxygen and nitrogen species, or a combination thereof. By way of non-limiting example, the waste molecules may comprise ammonia, ammonium, and/or lactate.

In some embodiments, a culture medium of cells or tissues is rejuvenated, wherein tissues are cultured for cultured meat production in at least one container, e.g., a bioreactor. Through the separation, removal and rejuvenating processes, the waste molecules that interfere with the proper growth of the cultured meat and/or that cause cell death are removed from the culture medium, while nutrients needed for the proper growth of the cultured meat are retained in the culture medium.

For the cell culture rejuvenating systems disclosed above and herein, the resultant rejuvenated medium may contain one or more proteins selected from the group consisting of albumin, catalase, insulin, fibroblast growth factor, transforming growth factor beta, and super oxide dismutase. These protein(s) are essential for cell growth and/or differentiation and are circulated back into the bioreactor for continuous use.

Additionally, in some embodiments, the resultant rejuvenated medium may contain one or more amino acids selected from the group comprising/consisting of glycine, serine, valine, threonine, isoleucine, leucine, asparagine, glutamine, lysine, methionine, histidine, phenylalanine, arginine, tyrosine, tryptophan and cysteine. Retention of the one or more amino acids in the rejuvenated media may be essential for cell growth in the rejuvenated media.

In some embodiments, the cell retention device may comprise at least one hollow fiber with a pore cutoff between about 5 kD and about 5 microns. By way of non-limiting example, the at least one hollow fiber has a pore cutoff of about 3 microns. In some embodiments, the cell retention device may comprise continuous centrifuge. By way of non-limiting example, the centrifuge may operate at 600 to 20,000×g, in some particular embodiments, the centrifuge may operate at 8400×g, thereby removing cells from the cell culture medium and creating a waste medium devoid of cells for rejuvenation.

After the separation, removal and rejuvenating processes, the system disclosed above and herein provides a rejuvenated medium that may comprise less than 30%, e.g., less than 20%, less than 10%, less than 5%, less than 2% or any intermediate, smaller or larger percentage value of waste molecules compared to the amount of waste molecules in the culture medium entering the system. In some embodiments, the rejuvenated medium may comprise more than 60%, e.g., more than 70%, more than 80%, more than 90%, more than 95% or any intermediate, smaller or larger percentage value of selected nutrients or other essential materials compared to the amount of the selected nutrients or other essential materials in the culture medium entering the system.

In some embodiments, ED may be combined with nanofiltration as disclosed above and herein, where the nanofiltration permeate is polished by ED to improve lactate and ammonium ion removal while maintaining high concentration of nutrients. The combination of nanofiltration with ED provides several additional benefits, when compared to nanofiltration alone. Polishing the nanofiltration permeate by ED increases the nutrient retention, by using high nanofiltration concentration factor and high inhibitors reduction. For example, the lactate and ammonium reduction using nanofiltration is up to 50%, while it goes up to 75% or more using integrated process of nanofiltration and ED. Also, polishing the nanofiltration permeate reduces volume (water) loss. In some embodiments, the volume loss using nanofiltration is 50%. In comparison the volume loss of the integrated process of nanofiltration and ED is only about 5%. For example, in the integrated system, less than 20% of the stream is concentrated, while almost about 50% of stream is concentrated with nanofiltration alone. Additionally, this lost volume from concentration contains amino acids and other essential growth nutrients. Water is also added back to dilute the nanofiltration concentrate, thus further increasing water usage. Thus, combining ED with nanofiltration can greatly improve the water and nutrient retention, while greatly enhancing the reduction in toxins from the rejuvenated media.

In some embodiments, the integrated system can reduce volume (water) loss by at least about 25% to about 90% in comparison to nanofiltration alone. In some embodiments, the integrated system reduces volume (water) loss by at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90% in comparison to nanofiltration alone. In some embodiments, water loss in an integrated system is in the range of about 5% or less to about 25% or less, or about 5%, or about 10%, or about 15%, or about 20%, or about 25% or less.

In some embodiments, the integrated system may result in a reduction of lactate in the rejuvenation media. In some embodiments, the reduction in lactate in the rejuvenated media in comparison to the input media is at least about 50% to at least about 95% of the input concentration. In some embodiments, the rejuvenated media has at least about 50%, or at least about 55%, or at least about 60%, or at least about 65%, or at least about 70%, or at least about 75%, or at least about 80%, or at least about 85%, or at least about 90%, or at least about 95% less lactate than input media.

In some embodiments, the integrated system may result in a reduction of ammonium in the rejuvenation media. In some embodiments, the reduction in ammonium in the rejuvenated media in comparison to the input media is at least about 50% to at least about 95% of the input concentration. In some embodiments, the rejuvenated media has at least about 50%, or at least about 55%, or at least about 60%, or at least about 65%, or at least about 70%, or at least about 75%, or at least about 80%, or at least about 85%, or at least about 90%, or at least about 95% less ammonium than the input media.

In some embodiments, wherein the integrated system is utilized, the combination may result in a reduction of additional waste products in the rejuvenation media. In some embodiments, the reduction in additional waste products in the rejuvenated media in comparison to the input media is at least about 50% to at least about 95% of the input concentration. In some embodiments, the rejuvenated media has at least about 50%, or at least about 55%, or at least about 60%, or at least about 65%, or at least about 70%, or at least about 75%, or at least about 80%, or at least about 85%, or at least about 90%, or at least about 95% less additional waste products than the input media.

In some embodiments, the rejuvenated media from the integrated media better at supporting cell proliferation in a bioreactor than waste media or nanofiltered medium. In some embodiments, the rejuvenated media can support the growth of at least 3×10⁶ additional cells for each mL of rejuvenated medium. In some embodiments, the cell proliferation in the rejuvenated media is at least about 20%-100% more than what is seen in waste media. In some embodiments, the cell proliferation in the rejuvenated media is at least about 5%-50% more than what is seen with rejuvenation media from nanofiltration alone. In some embodiments, the increased cell growth is due to inhibitor reduction while retaining the essential nutrients in the rejuvenated treatments.

In such integrated embodiments any suitable nanofiltration device and/or membrane may be used with the ED system disclosed herein. Non-limiting examples of suitable membranes that may be utilized for nanofiltration include halogenated compounds such as tetrafluoroethylene, tetrafluoroethylene copolymers, tetrafluoroethylene-perfluoroalkyl vinylether copolymers, polyvinylidene fluoride, polyvinylidene fluoride copolymers, polyvinyl chloride, polyvinyl chloride copolymers; polyolefins such as polyethylene, polypropylene and polybutene; polyamides such as nylons; sulfones such as polysulfones and polyether sulfones; nitrile-based polymers such as acrylonitriles; and styrene-based polymers such as polystyrenes. In some embodiments, the nanofiltration membrane as a molecular weight cutoff of from about 150 to about 300 Da, or about 150 Da to about 200 Da, or about 200 Da to about 250 Da, or about 250 Da to about 300 Da.

In some embodiments, the means for separating charged waste molecules from the cell culture medium may comprise an electrodialysis (ED) unit. In some embodiments, the ED unit may be a standard ED or a bi-polar ED (BPED). BPED may comprise a bi-polar membrane which allows for separate recovery of lactic acid and ammonium.

Careful choice of the voltage parameters for the ED unit to operate under is an important component for an effective medium rejuvenation system. In some embodiments, the ED unit may employ a voltage in the range of about 5-30 volts on the cell culture medium. By way of non-limiting example, the ED unit may employ about 0.1-4 Amperes on the cell culture medium.

Careful choice of the pH parameters of the cell culture medium is another important component for an effective medium rejuvenation system. In some embodiments, the cell culture medium may have a pH value in the range of about 3.8-8 when going through the ED process. In some embodiments, the cell culture medium may have a pH value in the range of about 6-8 when going through the ED process. By way of non-limiting example, the cell culture medium may have a pH value of about 7.

After the electrodialysis, the rejuvenated medium comprising salts and metals may be further processed at a pH range above 3.8, for example in the range of about 6-8. In some embodiments, the rejuvenated medium comprises iron and zinc may be adjusted to at least 0.05 mg/L before returning to the bioreactor.

Any of the systems described above and herein may further comprise a conductivity sensor to measure the osmolarity of the rejuvenated medium. In some embodiments, the osmolarity of the rejuvenated medium may be adjusted to be about 280-320 milliosmoles per kilogram (mOsm/kg) before returning to the bioreactor. By way of non-limiting example, the rejuvenated medium may have an osmolarity of about 290 mOsm/kg.

In some embodiments, the rejuvenated media, further corrected for osmolarity and salt as disclosed herein, is capable of supporting cell proliferation. In some embodiments, the rejuvenated media can support the growth of at least 3×10⁶ additional cells for each mL of rejuvenated medium.

For any of the systems of rejuvenating cell culture media described above and herein, biomass is expanded in the cell culture medium to produce edible/cultured meat. This system provides cost effective cell culture media for mass production of edible/cultured meat. In addition to edible/cultured meat, the system may be used for production of glycosylated proteins, viruses, genetic materials, or vaccines

Another embodiment of the present disclosure provides a method for rejuvenating a cell culture medium. Such method may comprise (a) obtaining a cell culture medium comprising waste molecules and essentially devoid of cells from at least one bioreactor using a cell retention device; (b) separating charged molecules from the cell culture medium obtained from (a) using an electric field, thereby removing the waste molecules from the cell culture medium and obtaining a rejuvenated medium essentially devoid of waste molecules; (c) retaining uncharged molecules and macromolecules in the rejuvenated medium during separation and removal of the charged molecules; and (d) circulating the rejuvenated medium back into the at least bioreactor or at least one other bioreactor, thereby rejuvenating the cell culture medium. In some embodiments, the method may further comprise a nanofiltration step wherein the culture medium from step (a) is passed through a nanofiltration means prior to ED as provided in steps (b) and (c).

In some embodiments, the cell culture medium obtained from (a) may further comprise one or more proteins. In some embodiments, the waste materials or waste molecules may be any materials or molecules that interfere with desired growth and/or desired differentiation of cells cultured in the cell culture medium. For instance, the waste materials or waste molecules may inhibit cell growth and/or differentiation or induce cell death. In some embodiments, the waste material(s) include, but are not limited to, ammonia, ammonium, lactate, sodium, isovaleric acid, isobutyric acid, 2-methylbutyric acid, rosmarinic acid, 3-phenyllactic acid, DL-p-Hydroxyphenyllactic acid, fumarate, alanine, glutamic acid, aspartic acid, reactive oxygen and nitrogen species, or a combination thereof. By way of non-limiting example, the waste molecules may comprise ammonia, ammonium, and/or lactate.

A cell culture medium comprises nutrients, essential materials, and waste materials, wherein separation is desired to remove the waste materials from the medium. For the methods of rejuvenating a cell culture medium disclosed above and herein, the culture medium of cells or tissues is rejuvenated, wherein tissues are cultured for cultured meat production in at least one container, e.g., a bioreactor. Through the separation, removal, and rejuvenating processes, the waste molecules that interfere with the proper growth of the cultured meat and/or that cause cell death are removed from the culture medium, while nutrients needed for the proper growth of the cultured meat are retained in the culture medium.

For the cell culture rejuvenating methods disclosed above and herein, the resultant rejuvenated medium comprises essential materials for cell growth and/or differentiation is circulated back into the bioreactor for continuous use. In some embodiments, the rejuvenated medium may contain one or more proteins selected from the group consisting of albumin, catalase, insulin, fibroblast growth factor, transforming growth factor beta, and super oxide dismutase.

In some embodiments, the cell retention device may comprise at least one hollow fiber with a pore cutoff between about 5 kD and about 5 microns. By way of non-limiting example, the at least one hollow fiber has a pore cutoff of about 3 microns.

In some embodiments, the cell retention device may comprise continuous or non-continuous centrifuge. By way of non-limiting example, the centrifuge may operate at 600 to 20,000×g, in some particular embodiments, the centrifuge may operate at 8400×g, thereby removing cells from the cell culture medium and creating a waste medium devoid of cells for rejuvenation.

In some embodiments, the electric field used to separate charged waste molecules from the cell culture medium may be provided by an electrodialysis (ED) unit. In some embodiments, the ED unit may be a standard ED or a bi-polar ED (BPED). BPED comprises a bi-polar membrane which allows for separate recovery of lactic acid and ammonium.

Voltage parameters for the ED unit to employ on the cell culture medium should be carefully chosen to ensure efficacy of the rejuvenation system. In some embodiments, the ED unit may employ a voltage in the range of about 5-30 volts on the cell culture medium. By way of non-limiting example, the ED unit may employ about 0.1-4 Amperes on the cell culture medium.

Careful choice of pH parameters of the cell culture medium is another important factor to consider in order to effectively separate and remove charged waste molecules from the culture medium. In some embodiments, the cell culture medium may have a pH value in the range of about 3.8-8 while going through the ED process. By way of non-limiting example, the cell culture medium may have a pH value of about 7.

After the electrodialysis, the rejuvenated medium comprising salts and metals may be further processed at a pH in the range of about 6-8. In some embodiments, the rejuvenated medium comprises iron and zinc adjusted to at least 0.05 mg/L before returning to the bioreactor.

Any of the methods described above and herein may further comprise measuring the osmolarity of the rejuvenated medium. In some embodiments, such measurement may be taken using a conductivity sensor. In some embodiments, the osmolarity of the rejuvenated medium may be adjusted to be about 280-320 milliosmoles per kilogram (mOsm/kg) before returning to the bioreactor. By way of non-limiting example, the rejuvenated medium may have an osmolarity of about 290 mOsm/kg.

After the separation, removal and rejuvenating processes, any of the methods disclosed above and herein provides a rejuvenated medium that may comprise less than 30%, e.g., less than 20%, less than 10%, less than 5%, less than 2% or any intermediate, smaller or larger percentage value of waste molecules compared to the amount of waste molecules in the culture medium entering the system. In some embodiments, the rejuvenated medium may comprise more than 60%, e.g., more than 70%, more than 80%, more than 90%, more than 95% or any intermediate, smaller or larger percentage value of selected nutrients or other essential materials compared to the amount of the selected nutrients or other essential materials in the culture medium entering the system.

For any of the methods of rejuvenating cell culture media described above and herein, the rejuvenated cell culture medium may be used to produce edible/cultured meat. In some embodiments, biomass is expanded in the cell culture medium to produce edible/cultured meat. These methods provide cost effective cell culture media for mass production of edible/cultured meat. In addition to edible/cultured meat, these methods may also be used for production of glycosylated proteins, viruses, genetic materials, or vaccines.

Some embodiments of the present disclosure provide a method for expanding cells in a bioreactor. This method may comprise culturing tissues in a cell culture medium comprising nutrients and waste molecules; and rejuvenating the cell culture medium according to any of the methods disclosed above and herein to reduce the amount of waste molecules or remove the waste molecules from the medium. In some embodiments, the expanded cells may be used to produce cultured meat, glycosylated proteins, viruses, genetic materials, or vaccines.

In some embodiments, the current disclosure also encompasses a rejuvenated cell culture medium, wherein the rejuvenated cell culture medium is obtained from the systems and methods provided herein. In some embodiments, the rejuvenated cell culture medium can be circulated to one or more bioreactors comprising cells or stored or packaged for future use.

In some embodiments, the current disclosure also encompasses a population of cultured cells, wherein the cultured cells are obtained by culturing cells in a rejuvenated cell culture medium obtained from the from the systems and methods provided herein. In some embodiments, the cultured cell or a population thereof may be a prokaryotic cell. In some embodiments, the cultured cell or a population thereof may be a eukaryotic cell. In some embodiments, the eukaryotic cell is a fungal cell (for example a yeast cell). In some embodiments, the eukaryotic cell is an avian cell, for example a chicken cell. In some embodiments, the eukaryotic cell is a mammalian cell, wherein the mammal may be, for example a bovine or a porcine. In some embodiments, the eukaryotic cell is a stem cell. In some embodiments, the cell is a somatic cell. In some embodiments, the eukaryotic cell is a collagen-secreting animal cells (e.g., fibroblasts, smooth muscle cells, etc.), from animals such as bovine, porcine, ovine, etc. In some embodiments, the cells may be sourced from live animals by biopsy or extracted from animals slaughtered for their meat. Alternatively, existing cell-lines (mammalian cell lines) may be used.

In some embodiments, the cells may be connective tissue cells. Connective tissue cells refers to the various cell types that make up connective tissue. In some embodiments, connective tissue cells are selected from fibroblasts, cartilage cells, bone cells, fat cells and smooth muscle cells. In some embodiments, connective tissue cells are selected from the group consisting of chondrocytes, adipocytes, osteoblasts, osteocytes, myofibroblasts, satellite cells, myoblasts and myocytes. In some embodiments, connective tissue cells are selected from the group consisting of, adipocytes, osteoblasts, osteocytes, myofibroblasts, satellite cells, myoblasts and myocytes. In some embodiments, connective tissue cells are fibroblasts. In some embodiments, the fibroblasts are not embryonic fibroblasts. In some embodiments, the fibroblasts are embryonic fibroblasts. In some embodiments, the fibroblasts are fetal fibroblasts. In some embodiments, the fibroblasts are dermal fibroblasts. In some embodiments, connective tissue cells are fibroblasts or a cell type that can be differentiated from a fibroblast. In some embodiments, connective tissue cells are not mesenchymal stem cells (MSCs). In some embodiments, connective tissue cells are not cells derived from MSCs. In some embodiments, connective tissue cells are cell that cannot be derived from MSCs. In some embodiments, the cell type can be naturally differentiated form a fibroblast. In some embodiments, the cell type results from natural fibroblast differentiation. As used herein, the “term natural differentiation” is used to refer to a differentiation that occurs in nature and not a trans-differentiation such as can artificially be achieved in a laboratory. In some embodiments, the natural differentiation is not de-differentiation. In some embodiments, a cell type that can naturally be differentiated form a fibroblast is selected from the group consisting of: a chondrocyte, an adipocyte, an osteoblast, an osteocyte, a myofibroblast, a myoblast and a myocyte. In some embodiments, a cell type that can naturally be differentiated form a fibroblast is selected from the group consisting of: an adipocyte, an osteoblast, an osteocyte, a myofibroblast, a myoblast and a myocyte. In some embodiments, a cell type that can naturally be differentiated form a fibroblast is an adipocyte. In some embodiments, the connective tissue cell is not a pluripotent cell. In some embodiments, the connective tissue cell is not a mesenchymal stem cell.

In some embodiments, the connective tissue cells are mammalian cells. In some embodiments, the mammal is a bovine. In some embodiments, the bovine is a cow. In some embodiments, the connective tissue cells are avian cells. In some embodiments, the connective tissue cells are fish cells. In some embodiments, the connective tissue cells are from an edible animal. In some embodiments, the cells are from livestock animals. In some embodiments, a livestock animal is selected from a cow, a pig, a goat, a sheep, a chicken, a fish and a turkey. In some embodiments, a livestock animal is selected from a cow, a pig, a goat, a sheep, a chicken, a fish, a duck, a goose and a turkey. In some embodiments, a livestock animal is selected from a cow, a pig, a goat, a sheep, a chicken, a duck, a goose and a turkey. In some embodiments, the connective tissue cells are selected from avian cells and bovine cells. In some embodiments, the bovine cells are cow cells. In some embodiments, the avian cells are chicken cells. In some embodiments, the connective tissue cells are selected from cow cells and chicken cells. In some embodiments, the chicken cells are chicken fibroblasts. In some embodiments, the cow cells are cow fibroblasts. In some embodiments, the chicken fibroblasts are DF-1 cells. In some embodiments, the cells are immortalized. In some embodiments, the cells are not immortalized. In some embodiments, the cells are derived from primary cells.

In some embodiments, the current disclosure also encompasses cultured meat and cultured meat products comprising cultured cells, wherein the cultured cells are obtained by from the system and methods provided herein. In some embodiments, the cultured meat may comprise additional components such as a plant protein.

In some embodiments, the current disclosure encompasses a cultured cell, wherein the cultured cell has been obtained by a method disclosed herein.

For example, in some embodiments, the current disclosure encompasses a cultured cell, wherein the cultured cell has been obtained by a method, wherein the method comprises: (a) obtaining a cell culture medium essentially devoid of cells from at least one bioreactor using a cell retention device, wherein the cell culture medium comprises waste molecules; (b) separating charged molecules from the cell culture medium obtained from (a) using an electric field, thereby removing the waste molecules from the cell culture medium and obtaining a rejuvenated medium essentially devoid of waste molecules; (c) retaining uncharged molecules and macromolecules in the rejuvenated medium during separation and removal of the waste molecules; (d) circulating the rejuvenated medium into the at least one bioreactor or at least one other bioreactor or combinations thereof; and (e) culturing the cells in the rejuvenated medium thereby obtaining the culture cell.

In some embodiments, the current disclosure encompasses a cultured cell, wherein the cultured cell has been obtained by a method, wherein the method comprises: (a) obtaining a cell culture medium essentially devoid of cells from at least one bioreactor using a cell retention device, wherein the cell culture medium comprises one or more waste molecules; (b) passing the cell culture medium from (a) through a nanofiltration mean, wherein the nanofiltration means is operable to provide a filtered permeate reduced in the one or more waste molecules; (c) separating charged molecules from the filtered permeate obtained from (b) using an electric field, thereby further reducing the one or more waste molecules from the cell culture medium and obtaining a rejuvenated medium essentially devoid of the one or more waste molecules; (d) retaining uncharged molecules and macromolecules in the rejuvenated medium during separation and removal of the one or more waste molecules; (e) circulating the rejuvenated medium back into the at least one bioreactor or at least one other reactor; and (f) culturing the cell in the rejuvenated medium, thereby obtaining the cultured cell.

In some embodiments, this disclosure encompasses a cultured cell, wherein the cultured cell has been cultured in a system disclosed herein.

For example, in some embodiments, the current disclosure encompasses a cultured cell, wherein the cultured cell has been cultured in a system, the system comprising: (a) at least one bioreactor comprising or configured to comprise one or more cells; (b) means for obtaining a cell culture medium essentially devoid of cells from the bioreactor using a cell retention device, wherein the cell culture medium comprises one or more waste molecules; (c) means for separating charged molecules from the cell culture medium obtained from (a) using an electric field, thereby removing the one or more waste molecules from the cell culture medium and obtaining a rejuvenated medium essentially devoid of one or more waste molecules; (c) means for retaining uncharged molecules and macromolecules in the rejuvenated medium during separation and removal of the charged molecules; and (d) means for circulating the rejuvenated medium back into the at least one bioreactor comprising or configured to comprise one or more cells or at least one other bioreactor comprising or configured to comprise one or more cells, thereby operable to produce the cultured cell.

In some embodiments, the current disclosure encompasses a cultured cell, wherein the cultured cell has been cultured in a system, the system comprising: (a) at least one bioreactor; (b) means for obtaining a cell culture medium essentially devoid of cells from the bioreactor using a cell retention device, wherein the cell culture medium comprises one or more waste molecules; (c) a nanofiltration means in fluid communication with the means in (a), wherein the nanofiltration means is operable to provide a filtered permeate reduced in the one or more waste molecules; (d) means for separating charged molecules from the filtered permeate obtained from (b) using an electric field, thereby further reducing the one of more waste molecules from the cell culture medium and obtaining a rejuvenated medium essentially devoid of waste molecules; (e) means for retaining uncharged molecules and macromolecules in the rejuvenated medium during separation and removal of the charged molecules; and (f) means for circulating the rejuvenated medium back into the at least one bioreactor comprising or configured to comprise one or more cells or at least one other bioreactor comprising or configured to comprise one or more cells, thereby operable to produce the cultured cell.

The following examples are offered by way of illustration and not by way of limitation.

### Example 1: Cell Culture Rejuvenation System Using Electrodialysis

Charged compounds can be separated from uncharged compounds through electrodialysis (ED). In order to recycle a cell culture medium for continuous use, charged waste molecules have to be separated and removed from the medium while uncharged materials essential for cell growth have to be retained in the medium. ED can separate and further remove lactate and ammonium in neutral pH from a cell growth medium as these compounds are charged, while most of the essential compounds in the cell growth medium are uncharged. Techniques such as nanofiltration may require pH adjustment for effective separation of lactate, which may cause denaturation of proteins. Therefore, nanofiltration usually requires separation of proteins with ultrafiltration (<5 kDa) as a prior treatment. ED provides a much-needed improvement in that it can effectively separate lactate and ammonium in neutral pH without the need of prior treatment of the growth medium to separate proteins. With this feature, the perfusion grade can be reduced to microfiltration instead of ultrafiltration. This is advantageous since the load on the perfusion can be reduced dramatically, which in turn increases the filtration flux by 10-folds.

Various medium rejuvenation systems are provided in the present disclosure, which employ ED to separate lactate and ammonium from a cell growth medium. As illustrated in FIGS. 2A-2B, a cell culture medium essentially devoid of cells is obtained from a bioreactor 1 using a cell retention device 2. Charged waste molecules are separated from the uncharged molecules and further removed from the cell culture medium by an electrodialysis unit (rejuvenation device) 3, while uncharged molecules are retained in the cell culture medium and further processed before recirculating back into the bioreactor 1. Additionally, macromolecules are also retained in the rejuvenated medium, although some may be charged. This retention is due to the size selectivity of the ED membranes. Cation exchange membrane (CEM) and anion exchange membrane (AEM) are homogenous gel-like membranes. They don't have pores similar to ultrafiltration membranes or others; however, the molecular structure of these membranes allows them to make a certain selectivity with respect to size (Ion Exchange Membranes for Electromembrane Process; PCA Technical Description; Nov. 20, 2019).

In some embodiments, the uncharged molecules are recycled to the bioreactor after salts and metals are compensated due to reduction in the ED. In some embodiments, after the removal, lactate and ammonium (the concentrate) may be further processed for recovery. For example, the base and the acid streams may be further treated with scrubber 7 to recover ammonium and with an ED to purify lactate in a process illustrated in FIG. 2C.

Since ED can effectively separate lactate and ammonium in neutral pH without the need of prior treatment of the growth medium to separate proteins, the perfusion grade can be reduced to microfiltration instead of ultrafiltration. This is true for both the standard ED (FIG. 2A) and BPED based cases (FIG. 2B).

A standard ED system is illustrated in FIG. 2A. Cells are grown in a bioreactor 1 and circulated through a cell retention device 2 with pores bigger than 50 kD allowing proteins such as albumin to be filtered out. A waste medium containing waste products and proteins but devoid of cells is removed from the cell retention device 2 and introduced to a rejuvenation device 3, wherein an electric field removes ammonia, lactate and other charged waste products to form a common waste stream 4. The remaining medium is depleted of waste products (see, a rejuvenated medium 5). The rejuvenated medium 5 is corrected for osmolarity and charged ions and subsequently circulated back to the bioreactor 1.

A rejuvenation system based on BPED is illustrated in FIG. 2B. Cells are grown in a bioreactor 1 and circulated through a cell retention device 2. A waste medium containing waste products but devoid of cells is removed from the cell retention device 2 and introduced to a rejuvenation device 3, wherein an electric field removes ammonia and other positively charged waste products to a base stream 4a, while lactate and other negatively charged waste products are removed to an acid stream 4b. The remaining medium is depleted of waste products (see, a rejuvenated medium 5). The rejuvenated medium stream 6 is corrected for osmolarity and charged ions and circulated back to the bioreactor 1.

As disclosed herein and above, the ED rejuvenation system comprises a bioreactor 1 for culturing the cells or tissues therein, a delivery means configured to deliver or feed a perfusion solution or cell culture medium to the bioreactor. The feeding is optionally and preferably continuous.

The rejuvenation system also comprises means 2 for removing a cell culture medium from the bioreactor 1, followed by means 3 for separating charged waste molecules from the cell culture medium, thereby removing the waste molecules from the cell culture medium and obtaining a rejuvenated medium essentially devoid of waste molecules. The waste medium contains waste materials/molecules that interfere with desired cell growth and/or differentiation and is essentially devoid of cells or large proteins, whereas the concentrate medium contains cells and other essential material(s) for cell growth and/or differentiation.

The means for removing a cell culture medium from bioreactor may be a cell retention device 2, which may comprise at least one hollow fiber with a pore cutoff of up to 5 microns. The porous walls act to prevent nutrients and other essential materials from crossing through. This is achieved by a porosity profile selected to provide optimal pore size and pore density. Each hollow fiber may be selected to have the same porosity profile. While the pores diameters (cut-off size) may not be constant, the pores diameter should on average be selected to prevent passage of high molecular weight materials, while permitting facile and efficient passage of small molecules, i.e., low molecular weight waste materials.

After the filtration, the waste medium that contains waste products is subjected to further processes while the remaining medium and cells are circulated back into the bioreactor. As illustrated therein, the system further comprises an ED unit 3 for separating charged waste molecules from the cell culture medium. After the ED, the charged waste materials are separated from the uncharged essential materials in the waste medium forming one or two waste streams, and a rejuvenated medium comprising the uncharged essential materials is obtained that is essentially devoid of any waste materials. This rejuvenation system involves operating ED within narrow pH and voltage parameters and actively correcting osmolarity and metal ion concentrations in the resultant rejuvenated medium.

The one or two waste streams may contain ammonia, ammonium salts, lactate, and/or amino acids of low molecular weight. In a standard ED system, one waste stream 4 is formed after ED (see, FIG. 2A), whereas in a BPED system, two waste streams 4a and 4b are formed after ED (see, FIG. 2B). The waste stream(s) may undergo further processes to isolate and recover individual components as illustrated in FIG. 2C. As provided in FIG. 2C, the waste stream may be pass through one or more of an ED unit 5, a BPED unit 6, a scrubber 7, and an extraction unit 8. The recovered individual components may possess commercial values that can be sold as individual products.

An additional rejuvenation system based on nanofiltration process and nanofiltration permeate polishing by a standard ED is illustrated in FIG. 2D. Cells are grown in a bioreactor 201 and circulated through a cell retention device 202. A waste medium containing waste products but devoid of cells is removed from the cell retention device 202 and introduced to a nanofiltration device 204, wherein the nanofiltration permeate, rich in lactate and ammonium and poor in amino acids, is introduced to an ED process 205, wherein an electric field removes ammonia, lactate and other charged waste products to form an ED concentrate stream 206. Uncharged molecules are remained in the ED diluate stream 207. The ED diluate 207 and the NF concentrate are merged (see, rejuvenated medium 208). The rejuvenated medium stream 208 is PH neutralized and corrected for osmolarity circulated to the bioreactor 201.

### Example 2: Rejuvenation Using Electrodialysis

### Materials and Methods

Standard Electrodialysis: The ED system used in the experiments was BED1-3 Compact Measurement (PCCEll GmbH, Germany). The standard ED cell unit comprised of 10 cell pairs of CEM and AEM. The CEM type used in the experiments was PC SK, while the AEM were either PC 100 D, PC 200 D or PC Acid 60. The membrane active area was 64 cm² (the system had 10 cell pairs, so the total membrane active area was 640 cm²). The power supplier operated up to 32 volts and up to 10 A with either constant voltage or constant current conditions. Each of the concentrate and diluate streams were recirculated in 10-100 L/hr, while the electrode rinse stream was recirculated 25-250 L/hr. The electrode rinse solution was 0.1 M sodium sulphate. The operation of the ED was initiated with 0.1 M sodium chloride. The conductivity and temperature of the concentrate and diluate streams were measured continuously.

Bi-polar electrodialysis: The ED system used in the experiments was based on the BED1-3 platform. The BPED cell unit comprised of 10 cell pairs. The CEM type used in the experiments was PC SK, while the AEM were either PC 100 D, PC 200 D or PC Acid 60. The membrane active area was 64 cm² cm² (the system had 10 cell pairs, so the total membrane active area was 640 cm²). The power supplier operated up to 32 volts and up to 10 A with either constant voltage or constant current conditions. Each of the acid, base and diluate streams were recirculated in 10-100 L/hr, while the electrode rinse stream was recirculated 25-250 L/hr. The electrode rinse solution was 0.1 M sodium sulphate.

The operation of the ED was initiated with 0.01 M sodium hydroxide and 0.01 M hydrochloric acid in the base and acid streams, respectively. The conductivity and temperature of the acid, base and diluate streams were measured continuously.

For both ED and BPED, the medium used in the experiments was cell growth medium consisting of essential growth factors for cell growth including, but not limited to, amino acids and vitamins. The medium was used in the diluate stream. During the ED and the BPED runs, samples were taken for chemical analysis (e.g., glutamine, glutamate, glucose, lactate, ammonium and osmolarity) in a chemistry analyzer (Accutrend Plus, Roche, Flex 2, Nova Biomedical) and for amino acids and vitamins analyses in a UPLC (Acquity, Waters).

### Results and Discussions

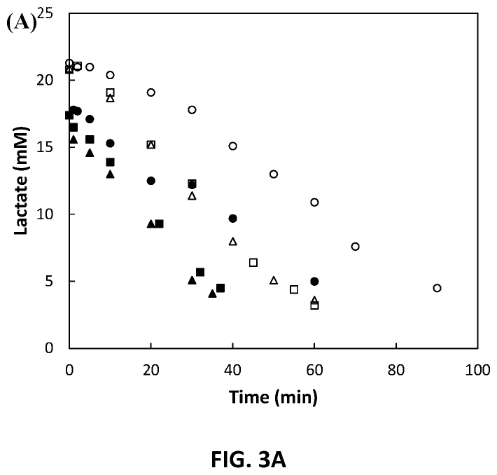

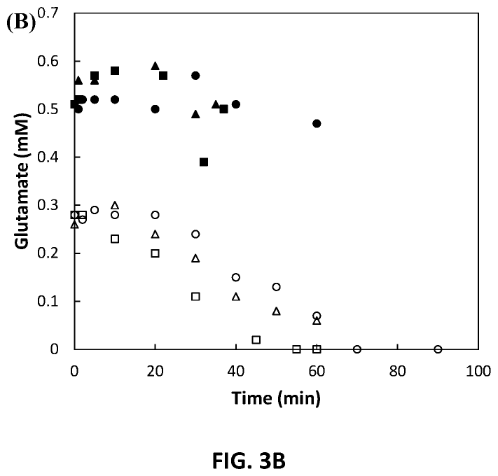

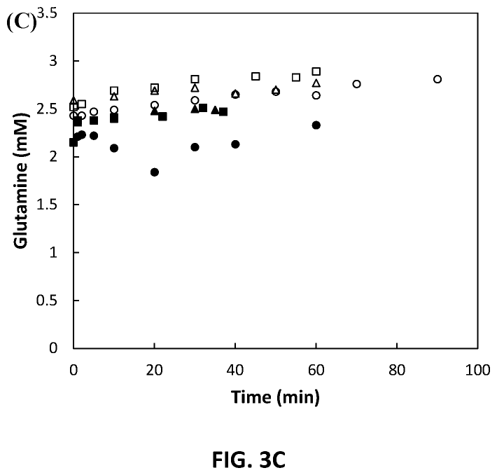

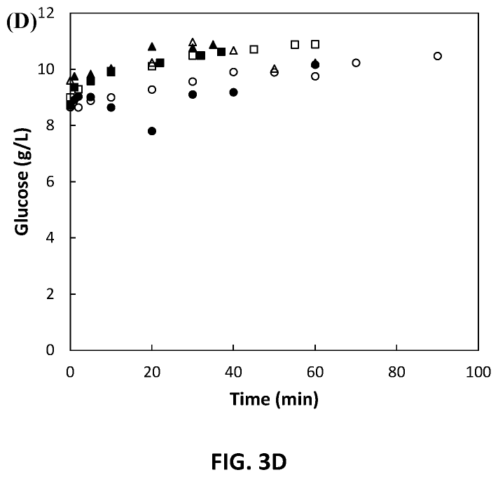

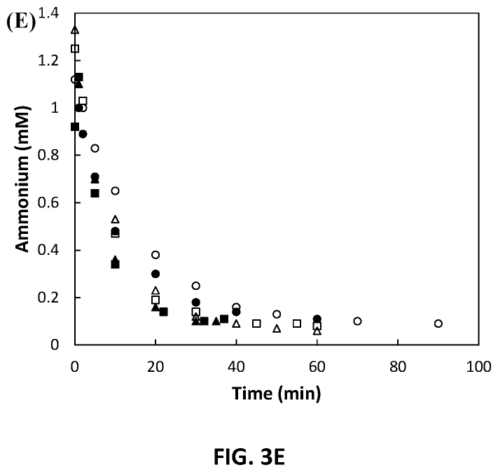

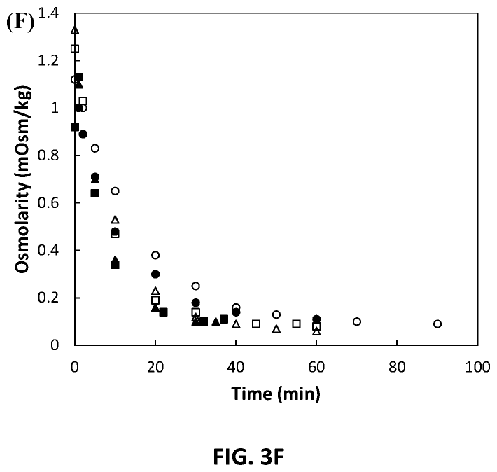

Lactate, ammonium, glucose, glutamine, glutamate and osmolarity reduction trends in a standard ED are illustrated in FIGS. 3A-3F. The lactate concentration decreased almost linearly overtime (FIG. 3A). The average lactate reduction rate using a PC 100 D AEM was 0.21, 0.35 and 0.37 mM/min for 8, 12 and 14 volts, respectively. The average lactate reduction rate using a PC 100 D AEM was 0.19, 0.31 and 0.31 mM/min for 8, 12 and 14 volts, respectively. The glutamate concentration reduction was negligible when the PC 100 D was used at any supplied voltage (FIG. 3B). The glutamate concentration reduction rate using a PC 200 D AEM was 3.5×10⁻³, 5.9×10⁻³ and 4.2×10⁻³ mM/min for 8, 12 and 14 volts. The glutamine and the glucose concentrations did not decrease at any supplied voltage or membrane (FIG. 3C and FIG. 3D, respectively). The ammonium and the osmolarity decreased exponentially overtime (FIG. 3E and FIG. 3F, respectively). The ammonium and the osmolarity values reached to saturation after approximately 30 minutes.

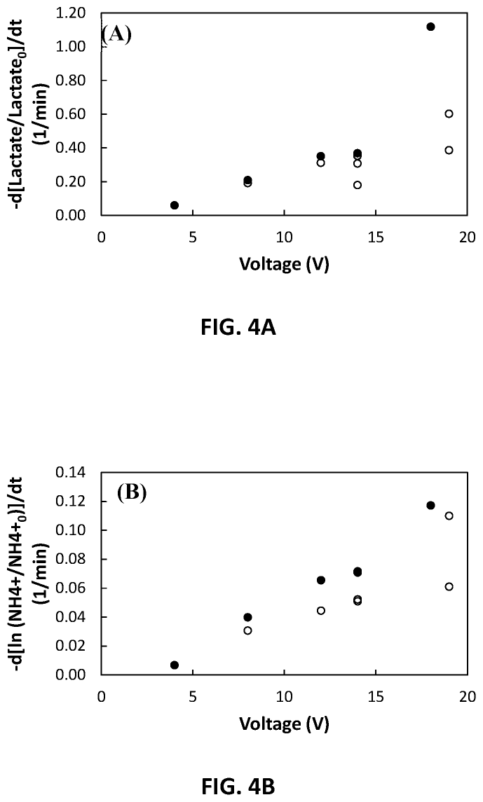

The reduction rate of the lactate using standard ED was presented as a function of the supplied voltage (FIG. 4A). The lactate reduction rate using PC 100 D was more linear as a function of voltage than the reduction using PC 200 D (R²=0.78 and R²=0.58, respectively). The reduction rate of the lactate using PC 100 D was almost the same as the reduction rate using PC 200 D up to 12 volts. Above 12 volts, the reduction rate using PC 100 D was faster.

The reduction rate coefficient of the ammonium, assuming exponential decrease, is presented in FIG. 4B. It is shown that the reduction rate coefficient of the ammonium using PC 100 D was slightly faster than the reduction rate using PC 200 D (9.1×10⁻⁴ 1/min, 2.11×10⁻³ 1/min and 2.07×10⁻³ 1/min).

The results show that small molecules permeate easily through the ED membranes. For example, the lactate (89 Da) concentration decreased linearly through ED (FIG. 3A). In comparison to lactate, the ammonium (18 Da) concentration, which is a smaller molecule, decreased exponentially (FIG. 3E). The osmolarity, which is mainly affected by the presence of sodium and chloride in the medium (23 Da and 35 Da, respectively) also decreased exponentially (FIG. 3F).

Both lactate and ammonium are charged in neutral pH; therefore, these compounds were collected in the concentrate stream in the experiments. The concentration of uncharged molecules such as glucose (180 Da) and glutamine (146 Da) did not decrease (FIG. 3C and FIG. 3D, respectively). Glutamate (148 Da), which is a charged amino acid, permeated to the concentrate stream using PC 200 Da. In contrast, the glutamine did not permeate using the PC 100 D. This difference is probably due to the difference between the MWCO of these membranes.

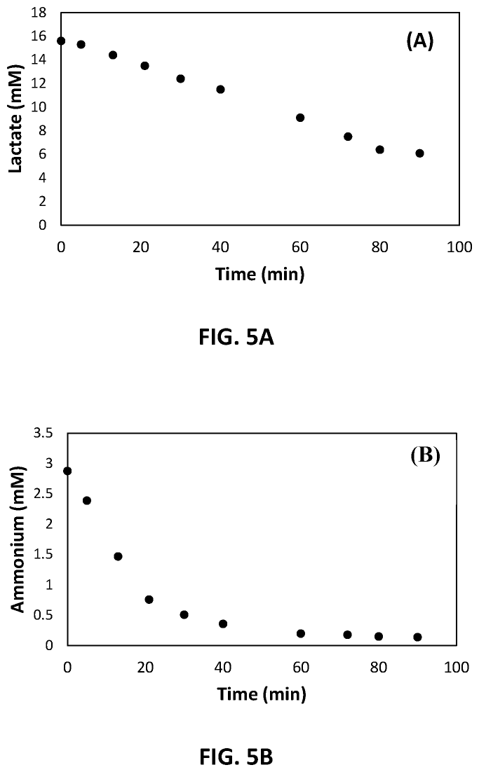

The reduction of the lactate and the ammonium concentration using BPED is illustrated in FIGS. 5A-5B. The lactate decreased almost linearly over time (0.11 mM/min, R²=0.996, FIG. 5A). The ammonium decreased exponentially over time, and reached to saturation after 60 minutes (FIG. 5B).

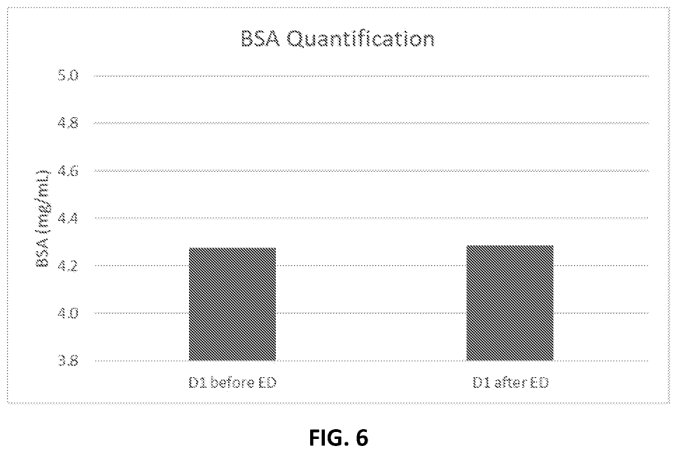

Quantification of bovine serum albumin (BSA) in the waste medium (before ED) and in the rejuvenated medium (after ED) during the BPED process was performed (see, FIG. 6). The results show no significant difference in BSA before and after ED, which suggests that no loss of protein occurred in the ED process.

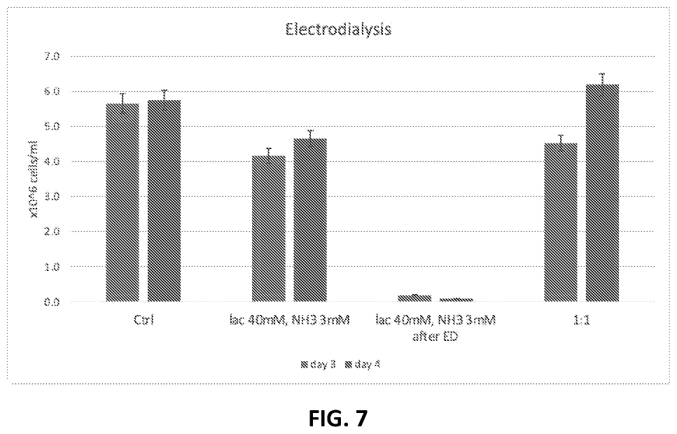

Effective cell proliferation is shown in rejuvenated media (see, FIG. 7). Standard culture medium (Ctrl) permits chicken cells to grow to 5-6×10⁶ cells/mL in 3-4 days. Waste media containing 40 mM lactate and 3 mM ammonia shows marginally lower proliferation reaching 4.5×10⁶ cells/mL (lac 40 mM, NH3 3 mM). Rejuvenated media after BPED does not support cells growth due to low osmolarity and salt content (after ED); however, rejuvenated media corrected for osmolarity and salts (1:1) support cell proliferation above 6×10⁶ cells/mL on Day 4.

### Example 3: Effect of Feed Source on Lactate Reduction

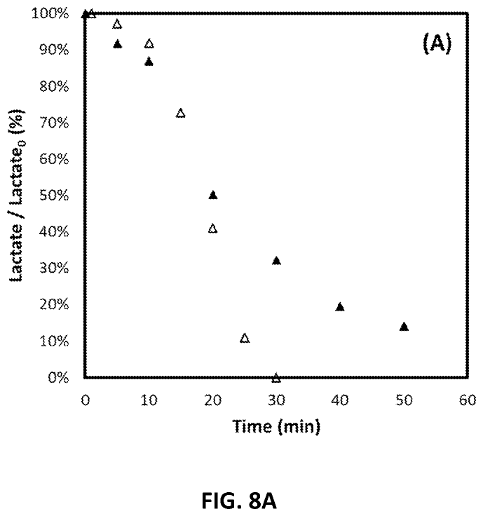

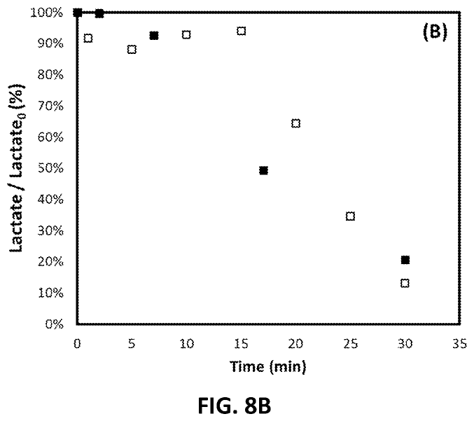

Tests were conducted to check for lactate reduction using two different feed source types, bioreactor waste and NF permeate (FIG. 8). In FIG. 8A, the ED treatment was carried with PC 200 D as an AEM. The initial phase of both treatments was an initial slow reduction phase (0-10 min, 1.3%/min and 0.8%/min for BR waste and NF permeate, respectively). This phase was followed by a drastic reduction phase (10-30 min, 2.75%/min and 4.6%/min for BR waste and NF permeate, respectively). The bioreactor waste source treatment showed additional slow reduction phase (from 30 min).

In FIG. 8B, the ED treatment was carried with PC 100 D as an AEM. The initial phase of both treatment was a lag phase (0-15 min and 0-7.5 min, for bioreactor waste and NF permeate, respectively). This phase was followed by a linear reduction phase (3.2%/min and 5.3%/min for BR waste and NF permeate, respectively).

From FIGS. 8A-8B, it can be understood that the membrane type effects the lactate recovery of media and permeate. In addition, the lag/initial slow phase is more pronounced using bioreactor waste than using NF permeate, and using PC 200 D than using PC 100 D. From the results shown in FIGS. 8A-8B, it can be deduced that ED treatment is more effective on NF's permeate than on media. The reason is probably the media contents vs permeate: the media is richer with solutes than the permeate, while lactate level is similar in both cases, and therefore the solutes are competitive to lactate molecules. Additionally, PC 200 D is more permeable than PC 100 D which permits the ions, such as lactate, to transport freely though the membranes.

### Example 4: Integrated Treatment Systems Using ED and Nanofiltration

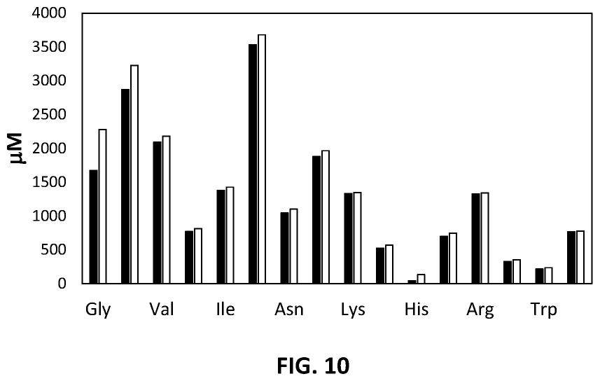

The cleaning factor of a rejuvenation treatment based on nanofiltration only is determined by the dilution factor of the concentrate stream, which ideally equals to the nanofiltration concentration factor. However, the nanofiltration concentration factor is limited by practical reasons (increased pressure). Moreover, the permeate stream contains some amount of essential nutrients. Therefore, increasing the nanofiltration volume concentration factor dilutes these compounds. Treating the nanofiltration permeate by ED (defined as nanofiltration polishing) can improve the rejuvenation process. Polishing the nanofiltration permeate by ED increases the nutrient retention, by using high nanofiltration concentration factor and high inhibitors reduction. For example, the lactate and ammonium reduction using nanofiltration only is up to 50%, while up to 75% using integrated process of nanofiltration and ED. Also, polishing the nanofiltration permeate reduces volume loss. For example, the volume loss using nanofiltration only is 50% (about 25% volume is required to be added by fresh water to dilute the nanofiltration concentrate). This volume contains amino acids and other essential growth nutrients (FIG. 10). In a comparison, the volume loss of integrated process of nanofiltration and ED is only 5%. In FIG. 10 the added amino acids are reflected by higher concentration at the integrated process than the nanofiltration only, that is because the nanofiltration permeate contains up to 10% amino acids, which in the nanofiltration only it goes to the drain and in the combined process the amino acids recycled. In addition, the only outlet stream in the rejuvenation process (besides the rejuvenated product) is in the ED stage (black box representation). To increase the global rejuvenation cleaning factor, one should enable increasing the NF permeate fraction, so in the ED stage, the inhibitors could be almost completely removed.

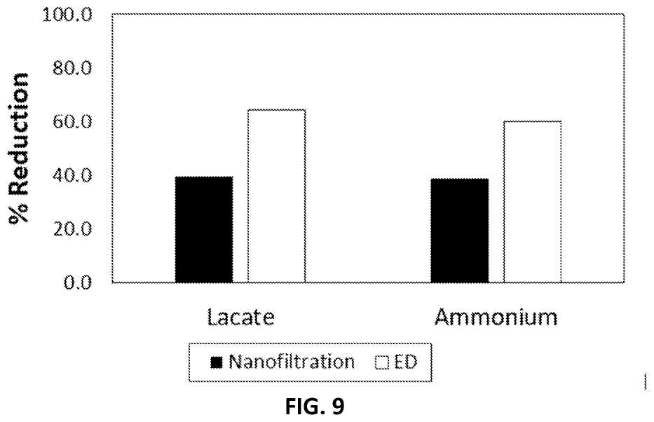

FIG. 9 demonstrates the inhibitors reduction using only nanofiltration and using integrated treatment of nanofiltration and ED. The lactate reduction was 40% for nanofiltration and 64% for integrated treatment of nanofiltration and ED. The ammonium reduction was 39% for nanofiltration and 60% for integrated treatment of nanofiltration and ED.

FIG. 10 demonstrates the increased recovery of amino acids when using ED as polishing treatment to the nanofiltration permeate. For example, glycine (75 Da) partially permeated the nanofiltration membrane (the concentration of glycine at the permeate was 52% of the waste concentration). Consequently, the glycine concentration at rejuvenation treatment using nanofiltration alone was only 74%. Polishing the permeate by standard ED recovered 100% of glycine from the bioreactor waste.

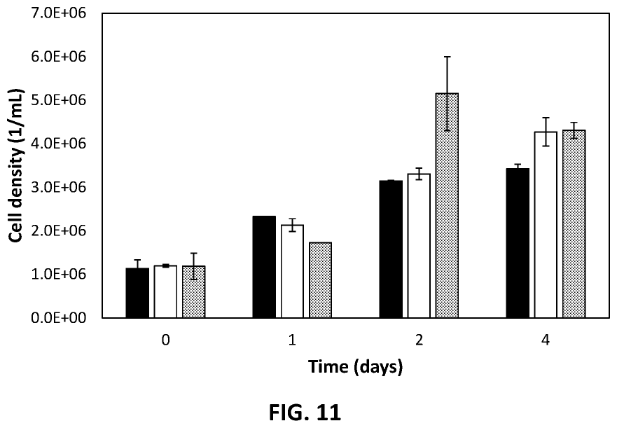

FIG. 11 shows cell proliferation in bioreactor waste medium and rejuvenated medium. The rejuvenated medium used only nanofiltration shows higher cell density than bioreactor medium since the second day of growth (for bioreactor waste medium: 3.15×10⁶ cells/mL and 3.43×10⁶ cells/mL at the second and fourth days of growth, respectively for nanofiltration: 3.31×10⁶ cells/mL and 4.27×10⁶ cells/mL at the second and fourth days of growth, respectively). The rejuvenated treatment based on integrated treatment of nanofiltration and ED showed slightly preference over nanofiltration only treatment from the second day of growth (5.15×10⁶ cells/mL and 4.31×10⁶ cells/mL at the second and fourth days of growth, respectively). The increased cell growth was due to inhibitor reduction while retaining the essential nutrients in the rejuvenated treatments.

### Example 5: Centrifuge Based Perfusion and Rejuvenation

In one example, FMT-SCF-4 chicken cells were seeded in a BioSTAT glass bioreactor at a density of 0.5×10⁶ cells/mL. Cells were grown in FX Serum-Free Medium to 3×10⁶ cells/mL at which point perfusion was initiated using GEA kytero 500 disk stack centrifuge working at continuous flow, or CARR UFMini non-continuous tubular centrifuge at 500 g. Waste media was collected from light phase and processed using nanofiltration to produce rejuvenated medium. Rejuvenated medium was further polished using electrodialysis (ED) as described above to produce rejuvenated medium that is capable of supporting up to 5×10⁶ additional cells per mL of medium.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A system for rejuvenating a cell culture medium, the system comprising:
(a) means for obtaining a cell culture medium essentially devoid of cells from at least one bioreactor using a cell retention device, wherein the cell culture medium comprises one or more waste molecules;
(b) means for separating charged molecules from the cell culture medium obtained from (a) using an electric field, thereby removing the one or more waste molecules from the cell culture medium and obtaining a rejuvenated medium essentially devoid of the one or more waste molecules;
(c) means for retaining uncharged molecules and macromolecules in the rejuvenated medium during separation and removal of the charged molecules; and
(d) means for circulating the rejuvenated medium back into the at least one bioreactor or at least one other bioreactor.

2. (canceled)

3. (canceled)

4. The system of claim 1, wherein the cell culture medium obtained from (a) further comprises one or more proteins.

5. The system of claim 1, wherein the one or more waste molecules interfere with desired growth and/or desired differentiation of the cells.

6. The system of claim 5, wherein the waste molecules include ammonia, ammonium, lactate, sodium, isovaleric acid, isobutyric acid, 2-methylbutyric acid, rosmarinic acid, 3-phenyllactic acid, DL-p-Hydroxyphenyllactic acid, fumarate, alanine, glutamic acid, aspartic acid, reactive oxygen and nitrogen species, or a combination thereof.

7. (canceled)

8. The system of claim 1, wherein the rejuvenated medium contains one or more proteins selected from the group consisting of albumin, catalase, insulin, fibroblast growth factor, transforming growth factor beta, and super oxide dismutase.

9. The system of claim 1, wherein the cell retention device comprises at least one hollow fiber with a pore cutoff between about 5 kD and about 5 microns, preferably about 3 microns.

10. The system of claim 1, wherein the cell retention device or cell retention centrifuge comprises continuous or dis-continuous centrifuge.

11. (canceled)

12. The system of claim 10, wherein the centrifuge operates at 300 to 20,000×g, preferably at 500×g, thereby removing cells from the cell culture medium and creating a waste medium devoid of cells for rejuvenation.

13. (canceled)

14. The system of claim 1, wherein the means for separating charged waste molecules from the cell culture medium comprises an electrodialysis unit, and wherein the electrodialysis unit employs a voltage in the range of about 5-20 volts on the cell culture medium.

15. (canceled)

16. The system of claim 1, wherein the cell culture medium has been adjusted to a pH value in the range of about 6-8.

17. (canceled)

18. The system of claim 1, wherein the rejuvenated medium has
(i) at least 50% to at least 80% lower ammonium that the cell culture medium, or
(ii) at least 50% to at least 80% lower lactate and/or ammonium than the cell culture medium.

19. (canceled)

20. The system of claim 14. wherein the electrodialysis unit comprises a bi-polar membrane which allows for separate recovery of lactic acid and ammonium.

21. The system of claim 1, wherein the rejuvenated medium comprising salts and metals is further processed at a pH in the range of about 6-8.

22. The system of claim 21, wherein the rejuvenated medium comprises iron and zinc adjusted to at least 0.05 mg/L before returning to the bioreactor.

23. The system of claim 1, further comprising a conductivity sensor to measure the osmolarity of the rejuvenated medium.

24. The system of claim 1, wherein the osmolarity of the rejuvenated medium is about 280-320 milliosmoles per kilogram (mOsm/kg) before returning to the bioreactor.

25. Use of the system of claim 1 for production of cultured cells, cultured meat, glycosylated proteins, viruses, genetic materials, or vaccines.

26. A method for rejuvenating a cell culture medium, the method comprising:
(a) obtaining a cell culture medium essentially devoid of cells from at least one bioreactor using a cell retention device, wherein the cell culture medium comprises one or more waste molecules;
(b) separating charged molecules from the cell culture medium obtained from (a) using an electric field, thereby removing the one or more waste molecules from the cell culture medium and obtaining a rejuvenated medium essentially devoid of one or more waste molecules;
(c) retaining uncharged molecules and macromolecules in the rejuvenated medium during separation and removal of the one or more waste molecules; and
(d) circulating the rejuvenated medium back into the at least one bioreactor or at least one other reactor, thereby rejuvenating the cell culture medium.

27. (canceled)

28. A method for efficient growth of eucaryotic cells for medical or agriculture applications, the method comprising:
(a) obtaining a cell culture medium essentially devoid of cells from at least one bioreactor using a cell retention centrifuge, thereby producing a waste medium essentially devoid of cells, wherein the waste medium is a cell culture medium comprising one or more waste molecules;
(b) collecting the waste medium and transferring the waste medium to a rejuvenation tank;
(c) controlling the pH of the waste medium from (b) prior to nanofiltration;
(d) passing the cell culture medium from (c) through a nanofiltration mean, thereby producing a permeate and a concentrated waste stream;
(e) removing one or more waste molecules from the permeate using an electric field. thereby producing a polished rejuvenated medium; and (f) recycling the concentrated waste and/or the polished rejuvenated medium back to the at least one means for growing eucaryotic cells, thereby increasing the efficient use of culture medium for eucaryotic cell growth.

29. The method of claim 26, wherein the cell culture medium obtained from (a) further comprises one or more proteins.

30. The method of claim 26, wherein the one or more waste molecules interfere with desired growth and/or desired differentiation of the cells.

31. The method of claim 30, wherein the waste molecules include ammonia, ammonium, lactate, sodium, isovaleric acid, isobutyric acid, 2-methylbutyric acid, rosmarinic acid, 3-phenyllactic acid, DL-/?-Hydroxyphenyllactic acid, fumarate, alanine, glutamic acid, aspartic acid, reactive oxygen and nitrogen species, or a combination thereof.

32. (canceled)

33. The method of claim 26, wherein the rejuvenated medium contains one or more proteins selected from the group consisting of albumin, catalase, insulin, fibroblast growth factor, transforming growth factor beta, and super oxide dismutase.

34. The method of claim 26, wherein the cell retention device comprises at least one hollow fiber with a pore cutoff between about 5 kD and about 5 microns.

35. The method of claim 26, wherein the cell retention device or cell retention centrifuge comprises continuous or non-continuous centrifuge.

36. The method of claim 35, wherein the centrifuge operates at 300 to 20,000×g, preferably at 500×g, thereby removing cells from the cell culture medium and creating a waste medium devoid of cells for rejuvenation.

37. The method of claim 26, wherein the electric field is provided by an electrodialysis unit, and wherein the electrodialysis unit employs a voltage in the range of about 5-30 volts on the cell culture medium.

38. (canceled)

39. The method of claim 26, wherein the cell culture medium has been adjusted to a pH value in the range of about 6-8.

40. (canceled)

41. The method of claim 37, wherein the electrodialysis unit comprises a bi-polar membrane which allows for separate recovery of lactic acid and ammonium.

42. (canceled)

43. (canceled)

44. The method of claim 26, further comprising measuring the osmolarity of the rejuvenated medium by a conductivity sensor.

45. The method of claim 26, wherein the osmolarity of the rejuvenated medium is about 280-320 milliosmoles per kilogram (mOsm/kg) before returning to the bioreactor.

46. The method of claim 26, wherein the rejuvenated cell culture medium is used for production of cultured meat, glycosylated proteins, viruses, genetic materials, or vaccines.

47. A method for expanding cells in a bioreactor, said method comprising:
(a) culturing cells in a cell culture medium comprising nutrients and waste molecules; and
(b) rejuvenating the cell culture medium according to the method of claim 1 to reduce the amount of the one or more waste molecules or remove the one or more waste molecules from the cell culture medium.

48. (canceled)

49. A rejuvenated cell culture medium, wherein the rejuvenated cell culture medium is obtained from the system of claim 1.

50. A rejuvenated cell culture medium, wherein the rejuvenated cell culture medium is obtained from the method of claim 26.

51. A cultured cell, wherein the cultured cell is obtained by culturing a cell in a rejuvenated cell culture medium obtained from the method of claim 26.

52. A cultured cell, wherein the cultured cell is obtained by culturing a cell in a system of claim 1.

53. A cultured meat comprising cultured cells, wherein the cultured cells are obtained by culturing cells in a system of claim 1.

54. A cultured meat comprising cultured cells, wherein the cultured cells are grown in rejuvenated cell culture medium obtained from the method of claim 26.

55. (canceled)

56. (canceled)

57. (canceled)

58. (canceled)
